(19) United States
(12) Patent Application Publication
(10) Pub. No.: US 2005/0233974 A1
(43) Pub. Date: Oct. 20, 2005

(54) Treating infectious diseases using ice inhibitors

(76) Inventors: John Randle, Brookline, MA (US); George Ku, Bedford, MA (US); Linda Hazlett, Grosse Pointe, MI (US)

Correspondence Address: VERTEX PHARMACEUTICALS INC., 130 WAVERLY STREET, CAMBRIDGE, MA, 02139-4242, US

(21) Appl. No.: 11/002,734

(22) Filed: Dec. 1, 2004

Related U.S. Application Data
(60) Provisional application No. 60/526,362, filed on Dec. 1, 2003.

(51) Int. Cl. A61K 38/05; A61K 38/04; A61K 31/5513

(52) U.S. Cl. 514/18; 514/19; 514/221

(57) ABSTRACT

This invention relates to methods and compositions for treating infectious and other diseases, particularly of the eye, by administering an ICE inhibitor. This invention also relates to methods for treating injuries, allergies, chemical irritations, or burns of the eye by administering an ICE inhibitor.

### CROSS-REFERENCE TO RELATED APPLICATION(S)

This application claims the benefit under 35 U.S.C. § 119 of U.S. Provisional Application 60/526,362, filed Dec. 1, 2003, the entire contents of that application being incorporated herein by reference.

### FIELD OF THE INVENTION

This invention relates to methods and compositions for treating infections and other diseases and disorders with an ICE inhibitor.

### BACKGROUND OF THE INVENTION

Pseudomonas aeruginosa (P. aeruginosa) keratitis is a sight threatening corneal disease that accounts for approximately ¾ of reported cases of contact lens-associated microbial infection (Liesegang, 1997). Disease progresses rapidly to cause ulceration of the cornea and can potentially lead to permanent loss of vision from corneal scaring if not treated aggressively (Laibson, 1972). Tissue damage during Pseudomonas keratitis can occur from multiple microbial (Engel et al., 1998; Kernacki et al., 1995) and host-associated factors (Steuhl et al., 1987; Steuhl et al., 1989). However, host inflammatory responses have been shown experimentally to play a critical role in the outcome of ocular infection with P. aeruginosa (Hazlett, 2002; Huang et al., 2002; Kernacki et al., 2000; McClellan et al., 2003; Rudner et al., 2000; Thakur et al., 2002; Xue et al., 2003a; Xue et al., 2003b).

Management of bacterial keratitis is intended to eliminate infectious organisms and to suppress the host's destructive inflammatory reaction. Conventionally, dual therapy (Baum and Barza, 2000; Dart and Seal, 1988; Guzek et al., 1994) with aminoglycosides and third-generation cephalosporins in fortified ophthalmic solutions or monotherapy (Leibowitz, 1991; Parks et al., 1993) with fluoroquinolones is prescribed for treating Pseudomonas keratitis. The recent increased incidence of refractory bacterial keratitis resulting from antibiotic (especially fluoroquinolone) resistant P. aeruginosa strains (Chaudhry et al., 1999; Garg et al., 1999; Kunimoto et al., 1999; Landman et al., 2002), is of great concern and also may limit future therapeutic choices.

Corticosteroids are a standard anti-inflammatory medication to treat residual inflammation with antibacterial therapy. At present, only corticosteroids are available in ophthalmic solutions to suppress the ongoing inflammatory response following bacterial corneal infection. However, identification of the causative organism and response to antibacterial therapy (or antibiotic sensitivity) are the key restrictive factors that must be considered before initiating corticosteroid therapy. The effect (beneficial or detrimental) of corticosteroids in reducing host mediated tissue damage has not been proven conclusively in bacterial keratitis (Hobden et al., 1993; Hobden et al., 1992; Phillips et al., 1983; Waterbury et al., 1987; Wilhelmus, 2002). Therefore, the controversial role of corticosteroids and emerging resistance of P. aeruginosa to antibiotics warrant development of new adjunctive therapeutic modalities.

Cytokines (especially IL-1β and TNF-α) are optimum therapeutic targets as they can initiate and sustain many diseases. Various strategies such as soluble receptors, antibodies, receptor antagonists or inhibitors are used to block cytokines. These specific anti-cytokine-based therapies have been shown to reduce inflammation in many chronic inflammatory or autoimmune diseases and are approved by FDA for human use (Bresnihan et al., 1998; Mohler et al., 1993; Nuki et al., 2002; van Deventer, 1999). The importance of IL-1β in the pathogenesis of Pseudomonas keratitis was demonstrated in previous studies (Rudner et al., 2000; Thakur et al., 2002; Xue et al., 2003b). Persistent elevated levels of IL-1β expression was associated with the severity of corneal disease, while reduced levels (after antibody neutralization or inhibition of IL-1β receptors) resulted in reduced disease severity.

Bacterial keratitis remains a major cause of sight-limiting scarring and visual impairment, especially in contact lens users (Poggio et al., 1989; Schein et al., 1989; Schein and Poggio, 1990), despite the efficacy of broad spectrum antibacterial agents. Over 30 million people use contact lenses in the United States alone (Barr et al., 2000), and 1 in 2,500 daily wear contact lens users and 1 in 500 extended wear contact lens users develop bacterial keratitis each year (Schein and Poggio, 1990). Traditionally, broad spectrum antibiotic (often ciprofloxacin) therapy is promptly instituted in keratitis cases after obtaining culture to identify a causative organism. Although anti-microbial treatment is often able to render a sterile cornea, it does not guarantee a clear visual axis, due to residual host derived inflammation. This may necessitate the use of corticosteroids to restore corneal clarity. In some cases, use of corticosteroids may have potential adverse effects, including delayed corneal wound healing (Leibowitz et al., 1996; Singh, 1985).

There is, therefore, a need for other approaches for treating inflammation in infections, particularly eye infections.

ICE, also known as caspase-1, is an intracellular protease that cleaves the precursors of IL-1β and IL-18 into active cytokines (Akita et al., 1997; Kuida et al., 1995). Although other proteases (including bacterial and host proteases) can process pro-IL-1β, ICE-deficient (ICE^{-/-}) mice have been shown incapable of releasing mature IL-1β in response to endotoxin (Fantuzzi et al., 1997; Li et al., 1995).

However, an ICE inhibitor has not been shown to be therapeutically effective in treating certain diseases, such as bacterial keratitis. There is, therefore, a need for small molecule ICE inhibitors for treating infections such as bacterial keratitis.

### SUMMARY OF THE INVENTION

The present invention relates to methods for treating infections and related disorders with an ICE inhibitor. This invention also relates to methods for treating injuries, allergies, chemical irritations, or burns of the eye by administering an ICE inhibitor.

Applicants have demonstrated the efficacy of an ICE inhibitor in experimental corneal infection induced by a clinical isolate of P. aeruginosa or a ciprofloxacin resistant P. aeruginosa strain. Clinical scores, histopathology, MPO activity, bacterial plate counts and ELISA analysis were used to assess the efficacy of treatment (at 18 h p.i.) with the ICE inhibitor vs. placebo +/− ciprofloxacin in C57BL/6 (B6) mice after corneal infection with P. aeruginosa strain 19660. Clinical scores were significantly reduced at 3, 5 and 7 days post-infection (p.i.) in the ICE inhibitor vs. placebo +/− ciprofloxacin treated mice. The decreased inflammatory response also was evidenced by reduced MPO activity and protein levels of IL-1β and MIP-2 at 7 days p.i. in the cornea of mice treated with an ICE inhibitor vs. placebo +/− ciprofloxacin.

Similarly bacterial load was reduced in the cornea at 7 days p.i. in mice treated with an ICE inhibitor vs. placebo without ciprofloxacin. An ICE inhibitor also reduced clinical scores after corneal infection induced by a clinical isolate-1025 or a ciprofloxacin resistant P. aeruginosa strain. Administration of an ICE inhibitor either alone or with ciprofloxacin significantly reduced corneal disease severity and show that the host inflammatory response after bacterial infection can be successfully managed by therapeutic strategies targeting ICE and IL-1β.

The present invention involves the use of inhibitors of ICE/caspase-1, whether selective for ICE/caspase-1, or broadly active on a range of other caspases (e.g., 2-14). This treatment, by inhibiting ICE and inhibiting IL-1β production will reduce the symptoms of infections and/or reduce the infection. In a preferred embodiment, the inhibitor is a selective ICE inhibitor.

The invention also relates to methods for identifying agents useful for treating these diseases.

The invention also relates to processes for preparing compositions and kits for practicing a method of this invention.

### DETAILED DESCRIPTION OF THE INVENTION

This invention provides methods for treating infections, particularly eye infections, by administering an ICE inhibitor.

Applicants have demonstrated that the use of an ICE inhibitor either alone or in combination with an antibiotic is very effective at treating keratitis in an animal model.

Specifically, applicants have demonstrated that an ICE inhibitor is able to control corneal degradation by regulating the host inflammatory response, as well as by the ability of the inhibitor to restrict bacterial growth, probably due to efficient bacterial killing in a reduced inflammatory milieu. The data provide evidence that reduction in endogenous IL-1β activity improves host defense against P. aeruginosa by down regulation of the tissue-damaging host derived inflammatory response.

Accordingly, one embodiment of this invention provides a therapeutic strategy for regulating a host inflammatory response.

Applicants have demonstrated surprisingly that the ICE inhibitor not only reduced inflammation in the model, but also reduced bacterial growth. Without being bound by theory, applicants believe ICE inhibitors reduce bacterial growth, partially because bacteria are not able to disseminate in a cornea in which damage is reduced.

An additional advantage of this invention is that ICE inhibitors can reduce symptoms such as pain, itchiness, and discomfort associated with various infections. Advantageously, these benefits can be achieved by using a single compound rather than by using multiple compounds (e.g., an antibacterial and an anti-inflammatory). Accordingly, this invention provides for the prevention, inhibition, control, termination, management, or reduction of virulence of microbial infections and/or inflammation and/or pain. In a preferred embodiment, the infection, inflammation, or pain is in the eye.

ICE inhibitor treated mice showed significantly reduced levels of both IL-1β and MIP-2 (chemoattractants for PMN), reduced PMN infiltration and bacterial load compared to a placebo treated group. Histopathological examination of the ICE inhibitor treated group showed markedly reduced infiltrating cells with intact corneal epithelium and supported the clinical score observations. Addition of the ICE inhibitor with topical antibiotic (ciprofloxacin) produced further improvement of corneal disease outcome.

Importantly, applicants have demonstrated that an ICE inhibition is efficacious not only against a standard ATCC laboratory strain (19660), but also against a clinical isolate (KEI-1025). Furthermore, ICE inhibition was found effective against a ciprofloxacin resistant strain derived from the parent 19660 strain. Clinical scores were significantly reduced in the ICE inhibitor treated corneas after infection with a ciprofloxacin resistant P. aeruginosa strain.

Various studies (Chaudhry et al., 1999; Garg et al., 1999; Kowalski et al., 2001; Kunimoto et al., 1999) have shown a link between in vitro antibiotic resistance and clinical failure to respond to antibiotic in keratitis patients. Garg et al., (1999) reported that of 141 culture-proven cases of Pseudomonas keratitis, 22 cases were caused by isolates resistant to ciprofloxacin (mean MIC 43 mg/ml). Of the 19 (of 22) cases treated initially with ciprofloxacin, 15 (76.7%) worsened or showed no clinical improvement after three days of intensive therapy and required modification of antibiotic therapy, corneal grafting or evisceration. Increasing incidence of antibiotic resistance of Pseudomonas and failure to respond to antibacterial therapy leading to adverse outcomes provide strong reasons to search for new therapeutic strategies. An ICE inhibitor could be a novel therapeutic strategy for antibiotic resistant Pseudomonas keratitis cases. Accordingly, this invention provides a method for controlling bacterial growth, especially in cases of microbial keratitis caused by an antibiotic resistant strain.

Infections by any microbial or pathogenic agent, such as those described in US 2004/0229802 (see particularly, paragraphs 0028-0039) may be treated in accordance with this invention. As would be realized, such agents can cause irritation, inflammation, redness, pain, tissue damage, and other adverse effects and symptoms. Thus, a method according to this invention may be used to ameliorate, treat, or prevent an infection, or symptoms thereof (including a bacterial infection, a viral infection, a parasitic infection, or a fungal infection) in a subject, comprising administering a compound that inhibits ICE to the subject. In a preferred embodiment, the method is for ameliorating, treating, or preventing an ocular infection. In a preferred embodiment, the method is for ameliorating, treating, or preventing keratitis (including infiltrative keratitis) or corneal ulcers. Other ocular diseases that would benefit from treatment according to this invention include, but are not limited to, those described in US 2004/0229802 (see particularly, paragraph 0025). Infections associated with contact lens use are also included (e.g., contact lens associated red eye (CLARE), contact lens induced peripheral ulcers (CLPU)).

In another embodiment, this invention provides a method for reducing bacterial growth in a subject comprising administering a compound that inhibits ICE to the subject. In another embodiment, this invention provides a method for co-administering an ICE inhibitor and an antimicrobial agent thereby reducing the bacterial growth in a subject.

In another embodiment, this invention provides a method for ameliorating, treating, or preventing an injury, allergy, chemical irritation, or burn of the eye in a subject, comprising administering to the subject a compound that inhibits ICE. In a preferred embodiment, this invention provides for promotion of healing of an eye injury and improved visual clarity. In a preferred embodiment, the eye injury is a corneal injury including, but not limited to, abrasions, lacerations, scratches, surgical trauma, accidental or incidental trauma, and bruises. In another embodiment, this invention provides a method for ameliorating, treating, or preventing dry eye (keratoconjunctivitis sicca), Sjogren's syndrome, aging of the eye comprising administering to the subject a compound that inhibits ICE. In another aspect, this invention could be used to ameliorate, treat, or prevent infections or adverse effects of inflammation or pain associated with eye surgery.

This invention is particularly useful for treating inflammation or reddening, of the superficial tissues of the eye. Eye disorders associated with inflammation include, for example, conjunctivitis (bacterial conjunctivitis, fungal conjunctivitis, or viral conjunctivitis), uveitis, keratic precipitates, macular edema, inflammatory response after intra-ocular lens implantation, and trauma caused by eye surgery or eye injury. In another embodiment, this invention provides methods of ameliorating, treating, or preventing these disorders.

Accordingly, this invention provides for ameliorating, treating, or preventing irritation, inflammation, redness, pain, tissue damage, and other adverse symptoms in the eye.

The compounds may be used to treat diseases and disorders, including infectious disease states, in subjects such as animals, preferably mammals, and more preferably humans. Methods of this invention can be used in veterinary settings involving zoo, laboratory, and farm animals. Accordingly, subjects include animals, such as primates, rodents, and birds, (including, but not limited to, guinea pigs, hamsters, gerbils, rat, mice, rabbits, dogs, cats, horses, pigs, sheep, cows, goats, rhesus monkeys, monkeys, tamarinds, apes, baboons, gorillas, chimpanzees, orangutans, gibbons, chickens, turkeys, ducks, geese, deer, and ostriches).

Any compound that inhibits ICE may be used in the methods and compositions of this invention. Such compounds include those compounds that inhibit ICE selectively and those that inhibit one or more enzyme in the caspase or ICE/CED-3 family. Such ICE inhibitors include, but are not limited to, the compounds described in WO 04/058718, WO 04/002961, WO 03/088917, WO 03/068242, WO 03/042169, WO 98/16505, WO 93/09135, WO 00/55114, WO 00/55127, WO 00/61542, WO 01/05772, WO 01/10383, WO 01/16093, WO 01/42216, WO 01/72707, WO 01/90070, WO 01/94351, WO 02/094263, WO 02/42278, WO 03/106460, WO 03/103677, WO 03/104231, U.S. Pat. No. 6,184,210, U.S. Pat. No. 6,184,244, U.S. Pat. No. 6,187,771, U.S. Pat. No. 6,197,750, U.S. Pat. No. 6,242,422, April 2001 American Chemical Society (ACS) meeting in San Diego, Calif., USA., WO 02/22611, US2002/0058630, WO 02/085899, WO 95/35308, U.S. Pat. No. 5,716,929, WO 97/22619, U.S. Pat. No. 6,204,261, WO 99/47545, and WO 01/90063 (which, as set forth herein, are all incorporated herein by reference). Preferred compounds for use in accordance with this invention are described in WO 04/058718, WO 04/002961, WO 95/35308, U.S. Pat. No. 5,716,929, WO 97/22619, U.S. Pat. No. 6,204,261, WO 99/47545, and WO 01/90063.

Included would be all isomeric (e.g., enantiomeric, diastereomeric, and geometric (or conformational)) forms of the structures; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the cited structure except for the replacement of hydrogen by deuterium or tritium, or the replacement of a carbon by a ¹³C- or ¹⁴C-enriched carbon are within the scope of this invention.

The compounds utilized in this invention may also be modified by appending appropriate functionalities to enhance selective biological properties. Such modifications are known in the art and include those which increase biological penetration into a given biological system (e.g., blood, lymphatic system, or central nervous system), increase oral availability, increase solubility to allow administration by injection, alter metabolism and/or alter rate of excretion.

More preferred compounds of this invention include: 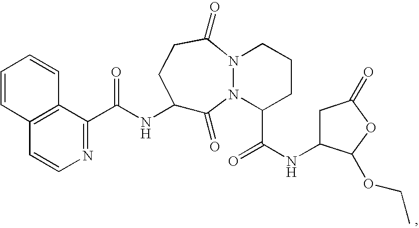
and each stereoisomer thereof, including: 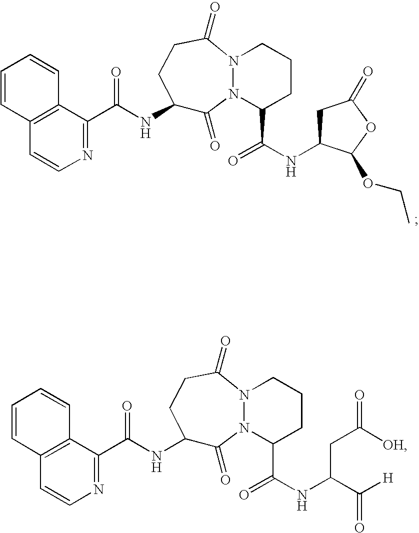
and each stereoisomer thereof, including: 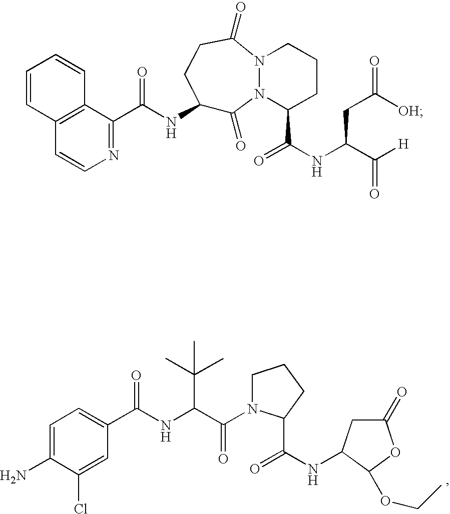
and each stereoisomer thereof, including: 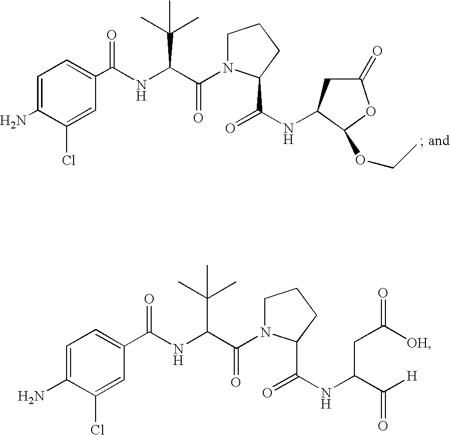
and each stereoisomer thereof, including:

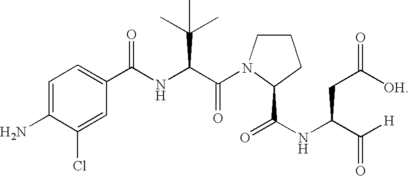

The compounds of this invention inhibit ICE and/or decrease IL-1, particularly IL-1β and IL-18 levels. These compounds can be assayed, for example, for their ability to inhibit the production of IL-1β and/or IL-18, regulate IL-1β and/or IL-18 levels, and/or affect IL-1β and/or IL-18 activity. Assays for each of the activities are known in the art, including those described below in detail in the Examples. Accordingly, these compounds are capable of targeting and inhibiting events in the ICE and/or IL-1β mediated diseases set forth herein.

This invention also provides methods for assaying compounds (ICE inhibitors) for anti-infective activity according to the methods herein and as known in the art.

The pharmaceutical compositions and methods of this invention, therefore, will be useful for controlling IL-1β levels and/or activity in vitro or in vivo. The compositions and methods of this invention will thus be useful for controlling IL-1β levels in vivo and for treating or reducing the advancement, severity or effects of certain conditions, including diseases, disorders, or effects as set forth herein.

According to another embodiment, the invention provides a composition comprising a compound of this invention (an ICE inhibitor) or a pharmaceutically acceptable derivative (e.g., salt) thereof, as described above, and a pharmaceutically acceptable carrier.

According to another embodiment, the compositions of this invention may further comprise another therapeutic agent. Such agents include, but are not limited to, a thrombolytic agent such as tissue plasminogen activator and streptokinase, an anti-inflammatory agent, a matrix metalloprotease inhibitor, a lipoxygenase inhibitor, a cytokine antagonist, an immunosuppressant, an anti-cancer agent, an anti-viral agent, a cytokine, a growth factor, an immunomodulator (e.g., bropirimine, anti-human alpha interferon antibody, IL-2, GM-CSF, methionine enkephalin, interferon alpha, diethyldithiocarbamate, tumor necrosis factor, naltrexone and rEPO), a prostaglandin, or an anti-vascular hyperproliferation compound. Other agents include, but are not limited to, one or more of the following: an additional ICE inhibitor, a NSAID (see, e.g., WO 01/08689), an antimicrobial agent, an antibacterial agent, an anti-inflammatory agent, and other agents, provided that they do not contradict the purpose of this invention (including, but not limited to, the agents described in US 2004/0191332, particularly at paragraphs 0042-0051 and WO 01/08689).

In pharmaceutical compositions comprising only a compound of this invention as the active component, methods for administering these compositions may additionally comprise the step of administering to the subject an additional agent, such as those described herein. When a second agent is used, the second agent may be administered either as a separate dosage form or as part of a single dosage form with the compounds or compositions of this invention.

The term “pharmaceutically acceptable carrier” refers to a non-toxic carrier that may be administered to a patient, together with a compound of this invention, and which does not destroy the pharmacological activity thereof.

Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The amount of compound present in the above-described compositions should be sufficient to cause a detectable decrease in the severity of the disease, or in ICE inhibition, IL-1 and/or IL-18 levels, or IL-1 and/or IL-18 activity.

If pharmaceutically acceptable salts of the compounds of this invention are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, the basic nitrogen-containing groups can be quaternized with such agents as lower alkyl halides, such as methyl, ethyl, propyl, and butyl chlorides, bromides and iodides; dialkyl sulfates, such as dimethyl, diethyl, dibutyl and diamyl sulfates; long chain halides such as decyl, lauryl, myristyl and stearyl chlorides, bromides and iodides; aralkyl halides, such as benzyl and phenethyl bromides and others. Water or oil-soluble or dispersible products are thereby obtained.

According to a preferred embodiment, the compositions of this invention are formulated for pharmaceutical administration to a subject, e.g., a mammal, preferably a human being.

Such pharmaceutical compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection and infusion techniques. Preferably, the compositions are formulated for administration to the eye.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or di-glycerides. Fatty acids, such as oleic acid and its glyceride derivatives are useful in the preparation of injectables, as are natural pharmaceutically-acceptable oils, such as olive oil and castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

If a solid carrier is used, the preparation can be tableted, placed in a hard gelatin capsule in powder or pellet form, or in the form of a troche or lozenge. The amount of solid carrier will vary, e.g., from about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

A syrup formulation can consist of a suspension or solution of the compound in a liquid carrier for example, ethanol, glycerin, or water with a flavoring or coloring agent. An aerosol preparation can consist of a solution or suspension of the compound in a liquid carrier such as water, ethanol or glycerin; whereas in a powder dry aerosol, the preparation can include e.g., a wetting agent.

Formulations of the present invention comprise an active ingredient together with one or more acceptable carrier(s) thereof and optionally any other therapeutic ingredient(s). The carrier(s) should be “acceptable” in the sense of being compatible with the other ingredients of the formulation and not deleterious to the recipient thereof.

The pharmaceutical compositions of this invention may be orally administered in any orally acceptable dosage form including, but not limited to, capsules, tablets, and aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

In ophthalmic compositions, the carriers should be ophthalmically acceptable, i.e., be a material that is compatible with ocular tissue at the concentration or amount in question. Such a material does not cause significant or undue detrimental effects when brought into contact with ocular tissues. Examples of such carriers are known to skilled practitioners (see, e.g., WO 01/08689 and US 2004/0229802). Aqueous carriers are preferred, particularly those that are at least about 50% by weight, water.

In ophthalmic compositions, the carrier may include one or more pharmaceutically or ophthalmically acceptable ingredients, such as tonicity (or isotonicity) adjusters, buffers, viscosity agents (e.g., thickeners), lubricants, surfactants, preservatives, emulsifiers, wetting agents, bodying agents, thixotropic agents, demulcents, and other components typically used in ophthalmic formulations. Examples of such carriers and ophthalmically acceptable ingredients are known to skilled practitioners (see, e.g., WO 01/08689, US 2004/0198763, US 2004/0191332, US 2004/0191332, and US 2004/0229802, which, as set forth herein, are all incorporated herein by reference).

Preferably, the pH of ophthalmic compositions is in the physiological range of the intended subject (e.g., about 3, 4 or 5 to about 7.5, 8.5, or 9, preferably about 7, about 7.5, or about 8).

An ophthalmic composition according to this invention may be in any form suitable for administration to the eye, such as solutions, suspensions, ointments, gels, and solids (see, e.g., WO 01/08689). Solid inserts and artificial tear compositions are included.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. In one embodiment, the compositions are as formulated herein. Other ophthalmic preparations may be found in, e.g., U.S. Pat. No. 6,645,994 and/or U.S. Pat. No. 6,630,473.

The pharmaceutical compositions of this invention may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents known in the art.

Composition of this invention may also include chelating or sequestering components or stabilizing agents (such as those described in WO 01/08689 and US 2004/0229802).

Compositions of this invention may be prepared according to conventional techniques. One embodiment of this invention provides a process for preparing an eye drop composition comprising combining an ICE inhibitor and a carrier (preferably sterile purified water) and optionally comprising combining an additional agent as set forth herein. Ophthalmic ointments may be prepared by combining an ICE inhibitor and a base (see, e.g., US 2004/0198763).

It will be recognized by one of skill in the art that the form and character of the pharmaceutically acceptable carrier or diluent is dictated by the amount of active ingredient with which it is to be combined, the route of administration, and other well-known variables.

Descriptions of the preparation and administration of ophthalmic and other formulations may be found in Remington: The Science and Practice of Pharmacy (formerly Remington's Pharmaceutical Sciences).

The above-described compounds and compositions are also useful in therapeutic applications relating to certain infectious diseases.

The compounds of this invention can inhibit the release IL-1β and/or IL-18 and thus can be useful for inhibiting or blocking several pathophysiological effects of certain diseases as set forth herein.

This invention also relates to a therapeutic method for treating certain diseases by (1) inhibiting IL-1β and/or IL-18 release from cells and/or (2) preventing the untoward, toxic or lethal effects of excessively high tissue levels of IL-1β and/or IL-18 in a mammal, including a human. This method comprises administering to a mammal an effective ICE inhibiting quantity of one or more ICE/CED-3 inhibitors. This method also can be used for the prophylactic treatment or prevention of certain diseases amenable thereto, including bacterial infections, viral infections, fungal infections, and parasitic infections. The invention provides a method for the treating these disorders by administering to a mammal, including a human, in need thereof an effective amount (i.e., therapeutically effective amount) of such compounds.

The compounds, by inhibiting ICE and blocking the release of IL-1β and/or IL-18 or decreasing IL-1β and/or IL-18 levels and activity, as well as the pathophysiologic actions of excessive levels of IL-1β and/or IL-18 in each of these circumstances, directly facilitate the arrest or resolution of certain diseases, and facilitates the restoration of normal function. Together, these actions relate their novel use in treating infectious diseases.

ICE inhibition may be measured by methods known in the art and as described more fully herein.

The compounds may be useful in inhibiting the release of IL-1β and/or IL-18 release by monocytes, macrophages, neuronal cells, endothelial cells, epidermal cells, mesenchymal cells (for example: fibroblasts, skeletal myocytes, smooth muscle myocytes, cardiac myocytes) and many other types of cells.

The term “condition” or “state” refers to any disease, disorder, or effect that produces deleterious biological consequences in a subject.

The level of IL-1β and/or IL-18 protein in the blood or cell of a patient or a cell culture (i.e., within the cell or the cell culture media) can be determined by for example, assaying for immunospecific binding to IL-1β and/or IL-18 or to other proteins known to be produced as a result of the presence of active IL-1β and/or IL-18. Such methods are known in the art. For example, immunoassays which can be used include, but are not limited to competitive and non-competitive assay systems, western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis with labeled antibodies. Such assays well known in the art (see, e.g., Ausubel et al, eds, 1994, Current Protocols in Molecular Biology, Vol. 1, John Wiley & Sons, Inc., New York, which is incorporated by reference herein in its entirety).

Competitive binding assays can also be used to determine the level of IL-1β and/or IL-18. One example of a competitive binding assay is a radioimmunoassay comprising the incubation of labeled proteins from cells expressing IL-1β (e.g., ³H or ¹²⁵I) with an anti-IL-1β antibody in the presence of increasing amounts of unlabeled IL-1β, and the detection of the anti-IL-1β antibody bound to the labeled IL-1β. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

IL-1β and/or IL-18 levels can also be assayed by activity, for example, IL-1β levels can be assayed by a cell line that is capable of detecting bioactive levels of cytokines like IL-1 or a growth factor. According to one embodiment, the levels of bioactive IL-1β in a biological sample is detected by incubating a cell line genetically engineered with isopropyl-b-D-thiogalactopyranoside. The cell line is incubated with the sample to be tested and cell death in the cell line is monitored by determining the intensity of blue color which is indicative of a bioactive cytokine or growth factor in the sample tested. [See also, e.g., X. -S. Liu, Burns 20(1), pp. 40-44 (1994) for TNF monitoring.]

Dosage levels of between about 0.01 and about 100 mg/kg body weight per day, preferably between about 0.5 and about 75 mg/kg body weight per day and most preferably between about 1 and about 50 mg/kg body weight per day of the active ingredient compound are useful in a monotherapy. Concentrations of ICE inhibitor between about 300 nM and 3 mM, preferably between about 3 μM and about 300 μM, would be useful in topical (e.g., eye drop) formulations. Such topical ophthalmic formulations will be administered as needed, preferably at a rate of about 1 to about 10 drops per eye and about 1 to about 10 times per day. In other ophthalmic formulation of this invention, an ICE inhibitor is present in an amount of at least about 0.001% (w/v or w/w), at least about 0.03% (w/v or w/w), at least about 0.15% (w/v or w/w) and in an amount of no more than about 10% (w/v or w/w), no more than about 3% (w/v or w/w), no more than about 1% (w/v or w/w) or no more than about 0.5% (w/v or w/w). A preservative, if present, is in an amount of at least about 0.0001 wt %, about 0.1 wt %, about 0.2 wt % to about 0.5 wt %, about 1 wt %, or about 2.5 wt %.

Typically, the pharmaceutical compositions of this invention will be administered from about 1 to 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

When the compositions of this invention comprise a combination of a compound of this invention and one or more additional therapeutic agents, both the compound and the additional agent should be present at dosage levels of between about 10% to about 80% of the dosage normally administered in a monotherapy regime.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained. When the symptoms have been alleviated to the desired level, treatment should cease. Patients may, however, require intermittent treatment on a long-term basis upon any recurrence or disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredients will also depend upon the particular compound and other therapeutic agent, if present, in the composition.

Accordingly, a method for treating or preventing a disease of this invention in a subject comprises the step of administering to the subject any compound, pharmaceutical composition, or combination described herein.

In a preferred embodiment, the invention provides a method of treating a mammal (preferably, a human being), having one of the aforementioned diseases, comprising the step of administering to said mammal a pharmaceutically acceptable composition described above. In this embodiment, if the patient is also administered another therapeutic agent, it may be delivered together with the compound of this invention in a single dosage form, or, as a separate dosage form. When administered as a separate dosage form, the other therapeutic agent may be administered prior to, at the same time as, or following administration of a pharmaceutically acceptable composition comprising a compound of this invention.

This invention also provides methods for assaying compounds (ICE inhibitors) for anti-infective activity according to the methods herein and as know in the art.

The methods for identifying a compound or composition for treating a disease according to this invention include methods for screening of a plurality of compounds or compositions for their ability to ameliorate the effects of certain disease(s) and/or improve the condition of a patient having certain disease(s) of this invention. According to one embodiment of this invention, high throughput screening can be achieved by having cells in culture in a plurality of wells in a microtiter plate, adding a different compound or composition to each well and comparing the ICE inhibition and/or IL-1β and/or IL-18 levels and/or activity in each cell culture to the levels or activity present in a cell culture in a control well. Controls that are useful for the comparison step according to this invention include cells or subjects that have not been treated with a compound or composition and cells or subjects have been treated with a compound or composition that is known to have no effect on ICE inhibition or activity.

According to one embodiment of this invention, the high throughput screening is automated so that the steps including the addition of the cells to the plate up to the data collection and analysis after addition of the compound or composition are done by machine. Instruments that are useful in the comparison step of this invention, e.g., instruments that can detect labeled objects (e.g., radiolabelled, fluorescent or colored objects) or objects that are themselves detectable, are commercially available and/or known in the art. Accordingly, compounds and compositions according to this invention that are useful for treating the certain disease disclosed herein can be quickly and efficiently screened.

One embodiment provides a method for identifying a compound that ameliorates, treats, or prevents an infectious disease (or other disease or disorder disclosed here), comprising contacting an infected cell population or cell culture with a compound that inhibits ICE and comparing the amount of infection in the cell population or cell culture to the amount infection in a cell population or cell culture that has not been treated with the ICE inhibitor.

Another embodiment provides a method for identifying a compound for ameliorating, treating, or preventing an infectious disease state in a subject comprising administering an ICE inhibitor according to any of WO 04/058718, WO 04/002961, WO 03/088917, WO 03/068242, WO 03/042169, WO 98/16505, WO 93/09135, WO 00/55114, WO 00/55127, WO 00/61542, WO 01/05772, WO 01/10383, WO 01/16093, WO 01/42216, WO 01/72707, WO 01/90070, WO 01/94351, WO 02/094263, WO 02/42278, WO 03/106460, WO 03/103677, WO 03/104231, U.S. Pat. No. 6,184,210, U.S. Pat. No. 6,184,244, U.S. Pat. No. 6,187,771, U.S. Pat. No. 6,197,750, U.S. Pat. No. 6,242,422, April 2001 American Chemical Society (ACS) meeting in San Diego, Calif., USA., WO 02/22611, US2002/0058630, WO 02/085899, WO 95/35308, U.S. Pat. No. 5,716,929, WO 97/22619, U.S. Pat. No. 6,204,261, WO 99/47545, and WO 01/90063 or a pharmaceutical composition comprising the compound and comparing the infectious disease state in the subject before and after treatment with the compound.

Another aspect of this involves a packaged kit for a patient to use in a treatment according to this invention, comprising: a single or a plurality of pharmaceutical formulation of each pharmaceutical component; a container housing the pharmaceutical formulation(s) during storage and prior to administration; and instructions for carrying out drug administration in a manner effective to carry out a method of this invention. Typically, such a kit will comprise, e.g. a composition of each ICE inhibitor and optionally the additional agent(s) in a pharmaceutically acceptable carrier (and in one or in a plurality of pharmaceutical formulations) and written instructions for the simultaneous or sequential administration. The kit may also comprise the ICE inhibitor in solid form and a pharmaceutically acceptable carrier and written instructions for preparing a pharmaceutical composition.

In another embodiment, a packaged kit is provided that contains one or more dosage forms for self administration; a container means, preferably sealed, for housing the dosage forms during storage and prior to use; and instructions for a patient to carry out drug administration. The instructions will typically be written instructions on a package insert, a label, and/or on other components of the kit, and the dosage form or forms are as described herein. Each dosage form may be individually housed, as in a sheet of a metal foil-plastic laminate with each dosage form isolated from the others in individual cells or bubbles, or the dosage forms may be housed in a single container, as in a plastic bottle. The present kits will also typically include means for packaging the individual kit components, i.e., the dosage forms, the container means, and the written instructions for use. Such packaging means may take the form of a cardboard or paper box, a plastic or foil pouch, etc.

All applications, patents and references disclosed herein are incorporated by reference.

In order that this invention be more fully understood, the following preparative and testing examples are set forth. These examples are for the purpose of illustration only and are not to be construed as limiting the scope of the invention in any way.

### EXAMPLES

### Example 1

Animal Infection

Eight week old female B6 mice (The Jackson Laboratory, Bar Harbor, Me.) were used in these experiments. The left cornea of each anesthetized mouse was scarified with three parallel 1 mm incisions using a sterile 25⅝ gauge needle under a stereoscopic microscope. Scarified corneas were challenged topically with 1.0×10⁶ CFU/μl of P. aeruginosa (ATCC strain 19660 or clinical isolate-1025 or a ciprofloxacin resistant 19660 strain in a 5 μl dose as described before (Kwon and Hazlett, 1997). Eyes were examined macroscopically at 1 day post-infection (p.i.) and at times described below to ensure that all mice were similarly infected and to monitor the course of disease. All animals were treated humanely and in full compliance with the Association for Research in Vision and Ophthalmology resolution on usage and treatment of animals in research.

### Example 2

Bacterial Strains

P. aeruginosa strain 19660 was used as a standard laboratory strain and produces reproducible corneal pathology in the B6 mouse model (kernacki et al., 2000; Rudner et al., 2000). P. aeruginosa strain 1025 (KEI-1025) was isolated in 1999 from a human microbial keratitis case at the Kresge Eye Institute, Detroit, Mich. The laboratory-derived ciprofloxacin-resistant mutant was developed by serially passaging the wild-type P. aeruginosa strain 19660 on ciprofloxacin-containing Luria-Bertani (LB) broth to obtain ciprofloxacin resistance (Sanchez et al., 2002). The ciprofloxacin-resistant P. aeruginosa strain when compared with the parent strain exhibited a 100-fold increase in the minimum inhibitory concentration (MIC) of ciprofloxacin (0.25 mg/ml vs. 25 mg/ml) required for in vitro killing of the bacteria. The virulence of the ciprofloxacin-resistant (P. aeruginosa-19660) mutant was decreased compared to the parent strain during the in vitro generation of this mutant, which is not unusual and has been reported previously (Bjorkman et al., 1998).

### Example 3

ICE Inhibitor Formulations

The ICE inhibitor used in these experiments displays potent inhibition of ICE (Ki=0.8 nM) and selectivity >100-fold vs. other non-ICE caspases. Four coded blinded formulations of vehicle (PBS) with or without ICE inhibitor (300 μM) were studied, for subconjunctival and topical administration. All formulations were found to be non-toxic to the eye in otherwise untreated mice and had no direct (in vitro) ability to kill bacteria.

### Example 4

Treatment Schedule

B6 mice (n=5/group) were injected subconjunctivally at 18 h p.i. with 5□□1 of 600 □M concentration of ICE inhibitor or placebo followed by topical application of D (ICE inhibitor) or C (placebo) +/− ciprofloxacin (Ciloxan; Alcon, Ft. Worth, Tex.) begun at 18 h p.i. and then 3×/day for 7 days. Initiation of ICE inhibitor therapy at 18 h p.i. was chosen to test experimentally in order to provide more clinically relevant data. In addition, by this time point, it would be expected that a patient would notice ocular symptoms such as haziness, discomfort and pain and would seek care.

### Example 5

Clinical Examination

Mice (n=5/group) were color coded and examined in masked fashion by two independent observers at 1, 3, 5 and 7 days p.i. to grade the severity of disease after P. aeruginosa infection. Ocular disease was graded and clinical scores were expressed using the following established scale (Hazlett et al., 1987): 0, clear or slight opacity partially covering the pupil; +1, slight opacity fully covering the anterior segment; +2, dense opacity partially or fully covering the pupil; +3, dense opacity covering the anterior segment; and +4, corneal perforation.

### Example 6

Histopathology

For Histopathological examination, eyes (n=3/group) from ICE inhibitor or placebo +/− ciprofloxacin treated mice were enucleated at 7 days p.i. Eyes were immersed in PBS, rinsed and placed in a fixative containing 1% osmium tetroxide, 2.5% glutaraldehyde, and 0.2M Sorenson's phosphate buffer (pH 7.4) in 1:1:1 ratio at 4° C. for 3 h. Eyes were transferred into a fresh fixative after 1.5 h and then dehydrated in graded ethanols, embedded in Eponaraldite, sections cut, stained with a modified Richardson's stain and photographed as described before (Hazlett et al., 2000).

### Example 7

Measurement of Myeloperoxidase (MPO) Activity

Samples were assayed for MPO activity as described before (Williams et al., 1982). Corneas (n=5/group) from ICE inhibitor or placebo +/− ciprofloxacin treated mice were collected at 7 days p.i. and homogenized in 1 ml of hexadecyl trimethylammonium bromide (HTAB) buffer (0.5% HTAB in 50 mM phosphate buffer, pH 6.0). The samples were subjected to three freeze-thaw cycles and then centrifuged at 14,000 rpm for 20 min. The supernatant was mixed with 50 mM phosphate buffer (pH 6.0), containing 0.167 mg/ml O-dianisidine hydrochloride and 0.0005% hydrogen peroxide at a 1:30 ratio in a total 3 ml volume. The change in absorbance at 460 nm was continuously monitored for 5 min. The results were expressed as units of MPO/cornea. One unit of MPO activity corresponds to approximately 2.0×10⁵ PMN cells (Williams et al., 1982).

### Example 8

Quantitation of Viable Bacteria in Cornea

At 7 days p.i., corneas (n=5/group) from ICE inhibitor or placebo +/− ciprofloxacin treated mice were collected and the number of viable bacteria was determined. For this, individual corneas were homogenized in sterile PBS and aliquots (100 μl) of serial dilutions were plated onto Pseudomonas isolation agar (Difco, Detroit, Mich.) plates in triplicate. Plates were incubated for 24 h at 37° C. Results were expressed as log₁₀ number of CFU/cornea±SEM.

### Example 9

Quantitation of Cytokine Proteins in Corneal Homogenate

Protein levels for IL-1β and MIP-2 were tested in ICE inhibitor or placebo +/− ciprofloxacin treated mice using ELISA kits (R & D Systems, Minneapolis, Minn.) per instructions of the manufacturer. Corneas (n=5/group) were removed at 7 days p.i. and immediately stored at −70° C. Before analysis, individual corneas were homogenized in 0.1% Tween 20-PBS with a glass Kontes pestle (Fisher, Itasca, Ill.) centrifuged at 5000×g for 10 minutes at 4° C., and supernatants were used to quantify IL-1β and MIP-2 proteins. Results are reported as pg/ml.

### Example 10

Statistical Analysis

The change in clinical score with time within a group was tested by the Friedman two-way analysis of variance by ranks. The difference in clinical score at each experimental time point between the ICE inhibitor and placebo treated B6 mice was tested by the Mann-Whitney U test. An unpaired, two-tailed Student's t-test was used to determine statistical significance for data from MPO assay, bacterial counts and ELISA analyses between treated and control groups. Mean differences were considered significant at P≦0.05. Experiments were repeated at least twice to ensure reproducibility and representative data from a single experiment are shown.

### Example 11

Results

ICE inhibitor-treated mice showed a significant decrease in disease severity at 3 (P=0.012), 5 (P=0.007) and 7 (P=0.007) days p.i. compared to vehicle-treated mice. Combined therapy with the ICE inhibitor and ciprofloxacin also resulted in significantly lower clinical scores at 5 (P=0.050) and 7 (P=0.047) days p.i. compared to the vehicle and ciprofloxacin-treated group. A significant difference also was observed in clinical scores within a group with time (vehicle, P=0.0001; vehicle+ciprofloxacin, P=0.009; ICE inhibitor, P=0.0014; ICE inhibitor+ciprofloxacin, P=0.77) except for the ICE inhibitor+ciprofloxacin treated group.

In the ICE inhibitor treated mice, a significant decrease in disease severity was found at 3 (P=0.007), 5 (P=0.015) and 7 (P=0.007) days p.i. (P. aeruginosa KEI-1025) compared to vehicle-treated mice. Similar to treatment with ICE inhibitor and ciprofloxacin in the P. aeruginosa strain 19660 infected corneas, this group (infected with KEI-1025) also showed significantly decreased clinical scores at 3 (P=0.007), 5 (P=0.015) and 7 (P=0.031) days p.i. in the B+D and ciprofloxacin treated corneas compared to vehicle plus ciprofloxacin-treated corneas. Clinical scores within a group with time also were significantly different (vehicle, P=0.0001; vehicle+ciprofloxacin, P=0.04; ICE inhibitor, P=0.0002; ICE inhibitor+ciprofloxacin, P=0.04).

Treatment of corneas infected with a ciprofloxacin resistant strain of P. aeruginosa (19660) with the ICE inhibitor showed significantly lower clinical scores at 3 (P=0.03), 5 (P=0.03) and 7 (P=0.007) days p.i. compared to the vehicle treated mice. Likewise, significantly lower clinical scores at 3 (P=0.03), 5 (P=0.03) and 7 (P=0.007) days p.i. were observed in the ICE inhibitor and ciprofloxacin treated group compared to the vehicle and ciprofloxacin treated group. The ICE inhibitor and ciprofloxacin treated mice showed similar clinical scores as ICE inhibitor vs. vehicle treated mice, confirming the in vivo resistance of this strain to ciprofloxacin. Clinical scores were significantly different with time in the vehicle (P=0.01) and ICE inhibitor (P=0.002) treated groups. No difference in clinical score was observed in the vehicle+ciprofloxacin, or ICE inhibitor+ciprofloxacin treated groups with time.

Slit lamp microscopy in the ICE inhibitor vs. vehicle treated groups at day 7 p.i. confirmed the clinical scores. In the ICE inhibitor treated cornea, noticeably less cellular infiltration, which mainly localized in the central cornea over the pupil, was observed. Opacity seen in the inferior corneal region is due to infiltrating cells that have gravity settled in the anterior chamber. In contrast, all mice treated with the vehicle exhibited perforated cornea. Slit lamp examination in the ICE inhibitor and ciprofloxacin treated corneas showed slight corneal opacity compared to vehicle and ciprofloxacin treated corneas which showed heavier cellular infiltration in the cornea as well as in the anterior chamber.

Histopathological examination of eyes after ICE inhibitor treatment showed markedly reduced infiltrating cells in the corneal stroma with a minimal anterior chamber inflammatory cell response. In contrast, the vehicle treated B6 mice showed a heavy cellular infiltrate in the cornea with complete denudation of the corneal epithelium, central stromal degradation, severe edema and a severe anterior chamber inflammatory cell response. Corneas treated with the ICE inhibitor and ciprofloxacin showed only few inflammatory cells along the corneal endothelium and in the anterior chamber compared to a typical eye treated with the vehicle and ciprofloxacin, which showed a heavier inflammatory cells infiltrate in the anterior chamber and adherent to the corneal endothelium.

Myeloperoxidase (MPO) activity was assayed to quantify PMN infiltration in the cornea of the ICE inhibitor vs. vehicle treated mice at 7 days p.i. ICE inhibitor treated mice showed a significantly lower (P=0.04) number of PMN compared to the vehicle treated mice. Similarly in the ICE inhibitor plus ciprofloxacin vs. vehicle plus ciprofloxacin treated mice, MPO activity was significantly reduced (P=0.0024) in cornea at 7 days p.i. The units of MPO activity per cornea (±SEM) may be depicted.

Viable bacterial plate counts were determined in cornea (n=5/group) from ICE inhibitor vs. vehicle treated groups at 7 days p.i. A significantly decreased (P=0.02) number of viable bacteria in the corneas of the ICE inhibitor treated group was found when compared to the placebo treated group. No bacterial colonies were isolated from plate counts in corneas from ICE inhibitor and ciprofloxacin and vehicle and ciprofloxacin treated groups at 7 days p.i. The mean log₁₀ number of viable bacteria per cornea (±SEM) may be depicted.

Protein levels of IL-1β and MIP-2 in the ICE inhibitor vs. vehicle treated groups were determined at 7 days p.i. using ELISA analysis. Significantly lower protein levels of IL-1β (P=0.023) and MIP-2 (P=0.012) were detected in the ICE inhibitor group compared to the vehicle treated group. Ciprofloxacin treatment markedly reduced the levels of both IL-1β and MIP-2, presumably due to a lessening of the pro-inflammatory stimulus by elimination of the infection. Nevertheless, in the ICE inhibitor and ciprofloxacin treated group, protein levels of IL-1β (P=0.036) and MIP-2 (P=0.04) also were further and significantly reduced compared to the vehicle and ciprofloxacin treated group.

### Example 12

ICE Inhibition

Compounds may be tested for their ability to inhibit ICE by methods known in the art (see, e.g., the documents cited herein).

### REFERENCES

Akita, K., Ohtsuki, T., Nukada, Y., Tanimoto, T., Namba, M., Okura, T., Takakura-Yamamoto, R., Torigoe, K., Gu, Y., Su, M. S., Fujii, M., Satoh-Itoh, M., Yamamoto, K., Kohno, K., Ikeda, M. and Kurimoto, M. (1997). Involvement of caspase-1 and caspase-3 in the production and processing of mature human interleukin 18 in monocytic THP.1 cells. J Biol Chem, 272, 26595-26603.
Barr, J. T., Zadnik, K., Wilson, B. S., Edrington, T. B., Everett, D. F., Fink, B. A., Shovlin, J. P., Weissman, B. A., Siegmund, K. and Gordon, M. O. (2000). Factors associated with corneal scarring in the Collaborative Longitudinal Evaluation of Keratoconus (CLEK) Study. Cornea, 19, 501-507.
Baum, J. and Barza, M. (2000). The evolution of antibiotic therapy for bacterial conjunctivitis and keratitis: 1970-2000. Cornea, 19, 659-672.
Bjorkman, J., Hughes, D. and Andersson, D. I. (1998). Virulence of antibiotic-resistant Salmonella typhimurium. Proc Natl Acad Sci USA, 95, 3949-3953.
Bresnihan, B., Alvaro-Gracia, J. M., Cobby, M., Doherty, M., Domljan, Z., Emery, P., Nuki, G., Pavelka, K., Rau, R., Rozman, B., Watt, I., Williams, B., Aitchison, R., McCabe, D. and Musikic, P. (1998). Treatment of rheumatoid arthritis with recombinant human interleukin-1 receptor antagonist. Arthritis Rheum, 41, 2196-2204.
Chaudhry, N. A., Flynn, H. W., Jr., Murray, T. G., Tabandeh, H., Mello, M. O., Jr. and Miller, D. (1999). Emerging ciprofloxacin-resistant Pseudomonas aeruginosa. Am J Ophthalmol, 128, 509-510.
Dart, J. K. and Seal, D. V. (1988). Pathogenesis and therapy of Pseudomonas aeruginosa keratitis. Eye, 2 Suppl, S46-55.
Engel, L. S., Hill, J. M., Moreau, J. M., Green, L. C., Hobden, J. A. and O'Callaghan, R. J. (1998). Pseudomonas aeruginosa protease IV produces corneal damage and contributes to bacterial virulence. Invest Ophthalmol Vis Sci, 39, 662-665.
Fantuzzi, G., Ku, G., Harding, M. W., Livingston, D. J., Sipe, J. D., Kuida, K., Flavell, R. A. and Dinarello, C. A. (1997). Response to local inflammation of IL-1 beta-converting enzyme-deficient mice. J Immunol, 158, 1818-1824.
Garg, P., Sharma, S. and Rao, G. N. (1999). Ciprofloxacin-resistant Pseudomonas keratitis. Ophthalmology, 106, 1319-1323.
Guzek, J. P., Chacko, D., Kettering, J. D., Wessels, I. F. and Aprecio, R. M. (1994). Comparison of topical ciprofloxacin to conventional antibiotic therapy in the treatment of experimental Pseudomonas aeruginosa keratitis.Cornea, 13, 500-504.
Hazlett, L. D. (2002). Pathogenic mechanisms of P. aeruginosa keratitis: a review of the role of T cells, Langerhans cells, PMN, and cytokines. DNA Cell Biol, 21, 383-390.
Hazlett, L. D., McClellan, S., Kwon, B. and Barrett, R. (2000). Increased severity of Pseudomonas aeruginosa corneal infection in strains of mice designated as Th1 versus Th2 responsive. Invest Ophthalmol Vis Sci, 41, 805-810.
Hazlett, L. D., Moon, M. M., Strejc, M. and Berk, R. S. (1987). Evidence for N-acetylmannosamine as an ocular receptor for P. aeruginosa adherence to scarified cornea. Invest Ophthalmol Vis Sci, 28, 1978-1985.
Hobden, J. A., Engel, L. S., Hill, J. M., Callegan, M. C. and O'Callaghan, R. J. (1993). Prednisolone acetate or prednisolone phosphate concurrently administered with ciprofloxacin for the therapy of experimental Pseudomonas aeruginosa keratitis. Curr Eye Res, 12, 469-473.
Hobden, J. A., O'Callaghan, R. J., Hill, J. M., Hagenah, M., Insler, M. S. and Reidy, J. J. (1992). Ciprofloxacin and prednisolone therapy for experimental Pseudomonas keratitis. Curr Eye Res, 11, 259-265.
Huang, X., McClellan, S. A., Barrett, R. P. and Hazlett, L. D. (2002). IL-18 contributes to host resistance against infection with Pseudomonas aeruginosa through induction of IFN-gamma production. J Immunol, 168, 5756-5763.
Kernacki, K. A., Barrett, R. P., Hobden, J. A. and Hazlett, L. D. (2000). Macrophage inflammatory protein-2 is a mediator of polymorphonuclear neutrophil influx in ocular bacterial infection. J Immunol, 164, 1037-1045.
Kernacki, K. A., Hobden, J. A., Hazlett, L. D., Fridman, R. and Berk, R. S. (1995). In vivo bacterial protease production during Pseudomonas aeruginosa corneal infection. Invest Ophthalmol Vis Sci, 36, 1371-1378.
Kowalski, R. P., Pandya, A. N., Karenchak, L. M., Romanowski, E. G., Husted, R. C., Ritterband, D. C., Shah, M. K. and Gordon, Y. J. (2001). An in vitro resistance study of levofloxacin, ciprofloxacin, and ofloxacin using keratitis isolates of Staphylococcus aureus and Pseudomonas aeruginosa. Ophthalmology, 108, 1826-1829.
G. Ku, A. Qadri and John C. R. Randle, (2001a) Interleukin-1β converting enzyme (caspase-1) inhibition with VX-765 reduces inflammation and cytokine levels in murine oxazolone-induced dermatitis. J. Invest. Dermatol. June issue, poster 856.
Ku, G., Qadri, A., and J. C. R. Randle (2001b) Interleukin-1β converting enzyme (ICE; caspase-1) inhibition with VX-765 reduces inflammation and cytokine levels in murine dermatitis and arthritis models.
Kuida, K., Lippke, J. A., Ku, G., Harding, M. W., Livingston, D. J., Su, M. S. and Flavell, R. A. (1995). Altered cytokine export and apoptosis in mice deficient in interleukin-1 beta converting enzyme. Science, 267, 2000-2003.
Kunimoto, D. Y., Sharma, S., Garg, P. and Rao, G. N. (1999). In vitro susceptibility of bacterial keratitis pathogens to ciprofloxacin. Emerging resistance. Ophthalmology, 106, 80-85.
Kwon, B. and Hazlett, L. D. (1997). Association of CD4+T cell-dependent keratitis with genetic susceptibility to Pseudomonas aeruginosa ocular infection. J Immunol, 159, 6283-6290.
   Laibson, P. R. (1972). Cornea and sclera. Arch Ophthalmol, 88, 553-574.
Landman, D., Quale, J. M., Mayorga, D., Adedeji, A., Vangala, K., Ravishankar, J., Flores, C. and Brooks, S. (2002). Citywide clonal outbreak of multiresistant Acinetobacter baumannii and Pseudomonas aeruginosa in Brooklyn, N.Y.: the preantibiotic era has returned. Arch Intern Med, 162, 1515-1520.
Leibowitz, H. M. (1991). Clinical evaluation of ciprofloxacin 0.3% ophthalmic solution for treatment of bacterial keratitis. Am J Ophthalmol, 112, 34S-47S.
Leibowitz, H. M., Bartlett, J. D., Rich, R., McQuirter, H., Stewart, R. and Assil, K. (1996). Intraocular pressure-raising potential of 1.0% rimexolone in patients responding to corticosteroids. Arch Ophthalmol, 114, 933-937.
Li, P., Allen, H., Banerjee, S., Franklin, S., Herzog, L., Johnston, C., McDowell, J., Paskind, M., Rodman, L., Salfeld, J. and et al. (1995). Mice deficient in IL-1 beta-converting enzyme are defective in production of mature IL-1 beta and resistant to endotoxic shock. Cell, 80, 401-411.
Liesegang, T. J. (1997). Contact lens-related microbial keratitis: Part I: Epidemiology. Cornea, 16, 125-131.
McClellan, S. A., Huang, X., Barrett, R. P., van Rooijen, N. and Hazlett, L. D. (2003). Macrophages restrict Pseudomonas aeruginosa growth, regulate polymorphonuclear neutrophil influx, and balance pro- and anti-inflammatory cytokines in BALB/c mice. J Immunol, 170, 5219-5227.
Mohler, K. M., Torrance, D. S., Smith, C. A., Goodwin, R. G., Stremler, K. E., Fung, V. P., Madani, H. and Widmer, M. B. (1993). Soluble tumor necrosis factor (TNF) receptors are effective therapeutic agents in lethal endotoxemia and function simultaneously as both TNF carriers and TNF antagonists. J Immunol, 151, 1548-1561.
Nuki, G., Bresnihan, B., Bear, M. B. and McCabe, D. (2002). Long-term safety and maintenance of clinical improvement following treatment with anakinra (recombinant human interleukin-1 receptor antagonist) in patients with rheumatoid arthritis: extension phase of a randomized, double-blind, placebo-controlled trial. Arthritis Rheum, 46, 2838-2846.
Parks, D. J., Abrams, D. A., Sarfarazi, F. A. and Katz, H. R. (1993). Comparison of topical ciprofloxacin to conventional antibiotic therapy in the treatment of ulcerative keratitis. Am J Ophthalmol, 115, 471-477.
Phillips, K., Arffa, R., Cintron, C., Rose, J., Miller, D., Kublin, C. L. and Kenyon, K. R. (1983). Effects of prednisolone and medroxyprogesterone on corneal wound healing, ulceration, and neovascularization. Arch Ophthalmol, 101, 640-643.
Poggio, E. C., Glynn, R. J., Schein, O. D., Seddon, J. M., Shannon, M. J., Scardino, V. A. and Kenyon, K. R. (1989). The incidence of ulcerative keratitis among users of daily-wear and extended-wear soft contact lenses. N Engl J Med, 321, 779-783.
Rudner, X. L., Kernacki, K. A., Barrett, R. P. and Hazlett, L. D. (2000). Prolonged elevation of IL-1 in Pseudomonas aeruginosa ocular infection regulates macrophage-inflammatory protein-2 production, polymorphonuclear neutrophil persistence, and corneal perforation. J Immunol, 164, 6576-6582.
Sanchez, P., Linares, J. F., Ruiz-Diez, B., Campanario, E., Navas, A., Baquero, F. and Martinez, J. L. (2002). Fitness of in vitro selected Pseudomonas aeruginosa nalB and nfxB multidrug resistant mutants. J Antimicrob Chemother, 50, 657-664.
Schein, O. D., Glynn, R. J., Poggio, E. C., Seddon, J. M. and Kenyon, K. R. (1989). The relative risk of ulcerative keratitis among users of daily-wear and extended-wear soft contact lenses. A case-control study. Microbial Keratitis Study Group. N Engl J Med, 321, 773-778.
Schein, O. D. and Poggio, E. C. (1990). Ulcerative keratitis in contact lens wearers. Incidence and risk factors. Cornea, 9 Suppl 1, S55-58; discussion S62-53.
Singh, G. (1985). Corticosteroids in corneal endothelial wound healing. Ann Ophthalmol, 17, 238-243.
Steuhl, K. P., Doring, G., Henni, A., Thiel, H. J. and Botzenhart, K. (1987). Relevance of host-derived and bacterial factors in Pseudomonas aeruginosa corneal infections. Invest Ophthalmol Vis Sci, 28, 1559-1568.
Steuhl, K. P., Doring, G. and Thiel, H. J. (1989). [The significance of bacterial and host factors in corneal infections caused by Pseudomonas aeruginosa]. Fortschr Ophthalmol, 86, 283-286.
Thakur, A., Xue, M., Stapleton, F., Lloyd, A. R., Wakefield, D. and Willcox, M. D. (2002). Balance of pro- and anti-inflammatory cytokines correlates with outcome of acute experimental Pseudomonas aeruginosa keratitis. Infect Immun, 70, 2187-2197.
van Deventer, S. J. (1999). Anti-TNF antibody treatment of Crohn's disease. Ann Rheum Dis, 58 Suppl 1, I114-120.
Waterbury, L., Kunysz, E. A. and Beuerman, R. (1987). Effects of steroidal and non-steroidal anti-inflammatory agents on corneal wound healing. J Ocul Pharmacol, 3, 43-54.
Wilhelmus, K. R. (2002). Indecision about corticosteroids for bacterial keratitis: an evidence-based update. Ophthalmology, 109, 835-842; quiz 843.
Williams, R. N., Paterson, C. A., Eakins, K. E. and Bhattacherjee, P. (1982). Quantification of ocular inflammation: evaluation of polymorphonuclear leucocyte infiltration by measuring myeloperoxidase activity. Curr Eye Res, 2, 465-470.
Xue, M. L., Thakur, A., Willcox, M. D., Zhu, H., Lloyd, A. R. and Wakefield, D. (2003a). Role and regulation of CXC-chemokines in acute experimental keratitis. Exp Eye Res, 76, 221-231.
Xue, M. L., Wakefield, D., Willcox, M. D., Lloyd, A. R., Di Girolamo, N., Cole, N. and Thakur, A. (2003b). Regulation of MMPs and TIMPs by IL-1 beta during corneal ulceration and infection. Invest Ophthalmol Vis Sci, 44, 2020-2025.

The documents cited herein are hereby incorporated by reference.

While we have described a number of embodiments of this invention, it is apparent that our basic examples may be altered to provide other embodiments, which utilize the compounds and methods of this invention. Therefore, it will be appreciated that the scope of this invention is to be defined by the appended claims rather than by the specific embodiments, which have been represented by way of example.

1. A method for treating an infection in a subject, comprising administering a compound that inhibits ICE to the subject.

2. The method according to claim 1, wherein the infection is a bacterial, viral, parasitic, or fungal infection.

3. The method according to claim 1 or claim 2, wherein the compound is administered with an additional agent selected from the group consisting of an antibiotic, antiviral, antiparasitic, or antifungal agent.

4. The method according to claim 1, wherein the infection is a bacterial infection.

5. The method according to claim 4, wherein the additional agent is an antibiotic agent.

6. The method according to any one of claims 1-5, wherein the infection is an eye infection.

7. The method according to claim 6, wherein the antibiotic, antiviral, antiparasitic, or antifungal agent is an ophthalmic drug.

8. A method for treating an injury, allergy, chemical irritation, or burn of the eye, dry eye, Sjogren's syndrome, aging of the eye in a subject, comprising administering to the subject a compound that inhibits ICE.

9. The method according to claim 8, wherein the compound is administered with an additional agent.

10. The method according to claim 9, wherein the additional agent is an ophthalmic drug.

11. The method according to claim 3 or claim 9, wherein the additional agent is administered with the compound in a single dosage form.

12. The method according to claim 3 or claim 9, wherein each additional agent is administered with the compound in separate dosage forms.

13. The method according to any one of claims 1-12, wherein the compound is formulated for administration to the eye.

14. The method according to any one of claims 1-6, wherein the compound is according to any of WO 04/058718, WO 04/002961, WO 03/088917, WO 03/068242, WO 03/042169, WO 98/16505, WO 93/09135, WO 00/55114, WO 00/55127, WO 00/61542, WO 01/05772, WO 01/10383, WO 01/16093, WO 01/42216, WO 01/72707, WO 01/90070, WO 01/94351, WO 02/094263, WO 02/42278, WO 03/106460, WO 03/103677, WO 03/104231, U.S. Pat. No. 6,184,210, U.S. Pat. No. 6,184,244, U.S. Pat. No. 6,187,771, U.S. Pat. No. 6,197,750, U.S. Pat. No. 6,242,422, April 2001 American Chemical Society (ACS) meeting in San Diego, Calif., USA., WO 02/22611, US2002/0058630, WO 02/085899, WO 95/35308, U.S. Pat. No. 5,716,929, WO 97/22619, U.S. Pat. No. 6,204,261, WO 99/47545, or WO 01/90063.

15. The method according to claim 14, wherein the compound is according to any of WO 04/058718, WO 04/002961, WO 95/35308, U.S. Pat. No. 5,716,929, WO 97/22619, U.S. Pat. No. 6,204,261, WO 99/47545, or WO 01/90063.

16. The method according to any one of claims 1-15, wherein the compound is selected from: 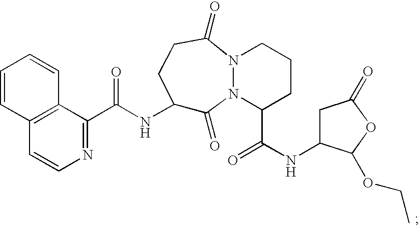
and each stereoisomer thereof, including:

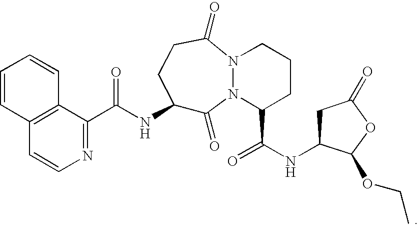

17. The method according to any one of claims 1-15, wherein the compound is selected from: 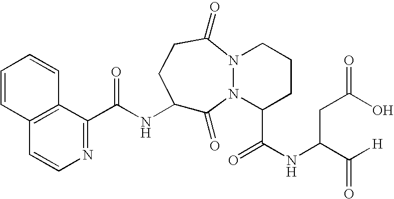
and each stereoisomer thereof, including:

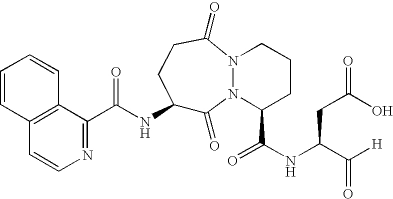

18. The method according to any one of claims 1-15, wherein the compound is selected from: 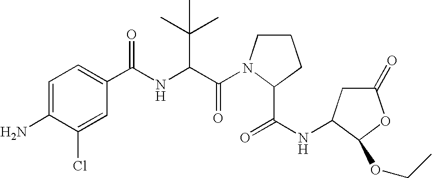
and each stereoisomer thereof, including:

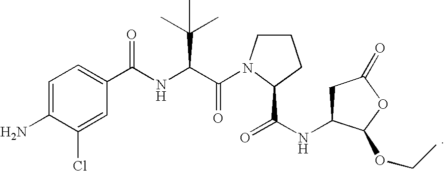

19. The method according to any one of claims 1-15, wherein the compound is selected from: 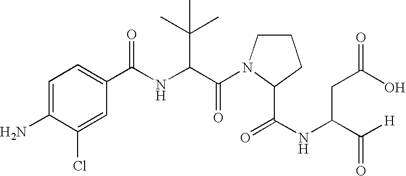
and each stereoisomer thereof, including:

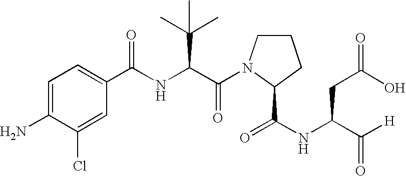

20. A pharmaceutical composition for ameliorating, treating, or preventing an infectious disease in a subject, comprising a compound that inhibits ICE and a pharmaceutically acceptable carrier.

21. The pharmaceutical composition according to claim 20, wherein composition further comprises an antibiotic, antiviral, antiparasitic, antifungal, or other ophthalmic drug.

22. The pharmaceutical composition according to claim 20 or claim 21, wherein composition is an ophthalmologic formulation.

23. An ophthalmic composition comprising and ICE inhibitor and a pharmaceutically acceptable carrier.

24. A pharmaceutical combination (or therapeutic combination) comprising an ICE inhibitor and an antibiotic, antiviral, antiparasitic, antifungal or other ophthalmic drug.

25. A pharmaceutical combination (or therapeutic combination) comprising an ICE inhibitor and an antibiotic.

26. A kit comprising an ICE inhibitor (and optionally an antibiotic, antiviral, antiparasitic, antifungal, or other ophthalmic drug) and instructions for treating an infection using the ICE inhibitor, the instructions optionally including instructions for administering an antibiotic, antiviral, antiparasitic, antifungal or other ophthalmic drug (whether or not included in the kit).
